(19) United States
(12) Patent Application Publication
(10) Pub. No.: US 2005/0143343 A1
(43) Pub. Date: Jun. 30, 2005

(54) Nutritional supplement for enhancing the production and effect of natural human growth hormone

(76) Inventor: Arnold P. Nerenberg, Whittier, CA (US)

Correspondence Address: ARLEN L. OLSEN;SCHMEISER, OLSEN & WATTS, 3 LEAR JET LANE, SUITE 201, LATHAM, NY, 12110, US

(21) Appl. No.: 10/748,615

(22) Filed: Dec. 30, 2003

(51) Int. Cl. A61K 31/715; A61K 31/4015; A61K 31/198

(52) U.S. Cl. 514/58; 514/423; 514/546; 514/564; 514/561; 424/439; 424/725; 424/535; 424/539; 514/78

(57) ABSTRACT

A nutritional supplement for enhancing the production and effect of natural human growth hormone. The nutritional supplement includes: L-arginine-2-pyrrolidone-5-carboxylate in an amount of about 500 milligrams to about 10 grams; L-lysine hydrochloride in an amount of about 500 milligrams to about 10 grams; and a cortisol suppressant including at least one of acetyl-L-carnitine in an amount of about 1 gram to about 10 grams and maltodextrin in an amount of about 1 gram to about 10 grams.

### BACKGROUND OF THE INVENTION

1. Technical Field

The present invention relates to a nutritional supplement for enhancing the production and effect of natural human growth hormone.

2. Related Art

Although existing nutritional supplements impact the production of natural human growth hormone, there is a need for an improved nutritional supplement that efficiently enhances the production and effect of natural human growth hormone.

### SUMMARY OF THE INVENTION

In first embodiments, the present invention provides a nutritional supplement, comprising:

L-arginine-2-pyrrolidone-5-carboxylate in an amount of about 500 milligrams to about 10 grams;

L-lysine hydrochloride in an amount of about 500 milligrams to about 10 grams; and

a cortisol suppressant including at least one of acetyl-L-carnitine in an amount of about 1 gram to about 10 grams and maltodextrin in an amount of about 1 gram to about 10 grams.

In second embodiments, the present invention provides a nutritional supplement consisting essentially of:

L-arginine-2-pyrrolidone-5-carboxylate in an amount of about 500 milligrams to about 10 grams;

L-lysine hydrochloride in an amount of about 500 milligrams to about 10 grams; and

a cortisol suppressant including at least one of acetyl-L-carnitine in an amount of about 1 gram to about 10 grams and maltodextrin in an amount of about 1 gram to about 10 grams.

In third embodiments, the present invention provides a nutritional supplement consisting essentially of:

L-arginine-2-pyrrolidone-5-carboxylate in an amount of about 500 milligrams to about 10 grams;

L-lysine hydrochloride in an amount of about 500 milligrams to about 10 grams;

a cortisol suppressant including at least one of acetyl-L-carnitine in an amount of about 1 gram to about 10 grams and maltodextrin in an amount of about 1 gram to about 10 grams; and

a substance consisting essentially of at least one of:
L-leucine in an amount of about 500 milligrams to about 10 grams,
bee pollen in an amount of about 50 milligrams to about 10 grams,
L-tyrosine in an amount of about 500 milligrams to about 10 grams,
taurine in an amount of about 500 milligrams to about 10 grams,
D-ribose in an amount of about 50 milligrams to about 10 grams,
colustrum in an amount of about 50 milligrams to about 10 grams,
whey protein in an amount of about 1 gram to about 20 grams,
lycopene in an amount of about 100 milligrams to about 10 grams,
glutamine in an amount of about 500 milligrams to about 10 grams, and
both macuna pruriens and alpha glycerylphosphorylcholine (GPC), said macuna pruriens in an amount of about 50 milligrams to about 5 grams, said alpha GPC in an amount of about 50 milligrams to about 5 grams.

The present invention advantageously provides a nutritional supplement that efficiently enhances the production and effect of natural human growth hormone.

### BRIEF DESCRIPTION OF THE DRAWINGS

FIGS. 1-5 depict structural forms of a nutritional supplement, in accordance with embodiments of the present invention.

### DETAILED DESCRIPTION OF THE INVENTION

The present invention discloses a nutritional supplement for use by a human being. In first embodiments, the nutritional supplement includes: L-arginine-2-pyrrolidone-5-carboxylate in an amount of about 500 milligrams to about 10 grams; L-lysine hydrochloride in an amount of about 500 milligrams to about 10 grams; and a cortisol suppressant comprising at least one of acetyl-L-carnitine in an amount of about 1 gram to about 10 grams and maltodextrin in an amount of about 1 gram to about 10 grams.

The combination of L-arginine-2-pyrrolidone-5-carboxylate and L-lysine hydrochloride is a blend of amino acids that induces the production of human growth hormone, as shown in a 1981 study at the University of Rome Medical Clinic V by Isidori et al. See A. Isidori M.D., A. Lo Monaco, M.D., and M. Cappa, “A Study of Growth Hormone Release in Man After Oral Administration of Amino Acids”, Current Medical Research and Opinion, Vol. 7., No. 7 (1981). The Isidori study used 15 male volunteers to evaluate the secretion of growth factors following oral administration of a combination of 1200 mg of L-arginine-2-pyrrolidone-5-carboxylate and 1200 mg of L-lysine hydrochloride. The results showed that human growth hormone was secreted in response to this stimulation. The study's result of human growth hormone release is specific to the combination of the two constituents (i.e., L-arginine-2-pyrrolidone-5-carboxylate and L-lysine hydrochloride). Neither of the two constituents resulted a significant release of human growth hormone when administered alone, even at the same doses. Increasing human growth hormone beneficially increases lean muscle mass, burns fat, boosts the immune system, revitalizes hair, nails, and skin, increases energy, strengthens tendons and ligaments, and enhances sexuality.

The nutritional supplement also includes a cortisol suppressant. Cortisol is produced by the adrenal glands in response to stimuli such as physical exercise, emotional stress, physical illness, etc. Cortisol facilitates the conversion of protein in muscles and connective tissue into glucose and glycogen. Thus, a cortisol suppressant in the nutritional supplement of the present invention functions synergistically with the combination of L-arginine-2-pyrrolidone-5-carboxylate and L-lysine hydrochloride. Whereas the combination of L-arginine-2-pyrrolidone-5-carboxylate and L-lysine hydrochloride promotes an increase in lean muscle mass, the addition of a cortisol suppressant serves to inhibit the breakdown of lean muscle mass. As stated supra, the cortisol suppressant of the nutritional supplement of the present invention comprises at least one of acetyl-L-carnitine in an amount of about 1 gram to about 10 grams and maltodextrin in an amount of about 1 gram to about 10 grams.

In addition to suppressing cortisol, acetyl-L-carnitine can boost energy and endurance to during exercise physical performance as well as provide other health benefits (e.g., combating stress and enhancing immunity).

In addition to suppressing cortisol, maltodextrin provides a sustained source of energy without increasing insulin levels. Thus maltodextrin provides slowly assimilated sugars to the body.

In addition to the L-arginine-2-pyrrolidone-5-carboxylate, L-lysine hydrochloride, and the cortisol suppressant, the nutritional supplement may include at least one of:

L-leucine in an amount of about 500 milligrams to about 10 grams;

bee pollen in an amount of about 50 milligrams to about 10 grams;

L-tyrosine in an amount of about 500 milligrams to about 10 grams;

taurine in an amount of about 500 milligrams to about 10 grams;

D-ribose in an amount of about 50 milligrams to about 10 grams;

colustrum in an amount of about 50 milligrams to about 10 grams;

whey protein in an amount of about 1 gram to about 20 grams;

lycopene in an amount of about 100 milligrams to about 10 grams;

glutamine in an amount of about 500 milligrams to about 10 grams, and

both macuna pruriens and alpha glycerylphosphorylcholine (“alpha GPC”), wherein the macuna pruriens is present in an amount of about 50 milligrams to about 5 grams, and wherein the alpha GPC is present in an amount of about 50 milligrams to about 5 grams.

L-leucine is a branched-chain amino acid that stimulates protein synthesis and therefore facilitates formation of muscle tissue. Thus, L-leucine functions synergistically with L-arginine-2-pyrrolidone-5-carboxylate, L-lysine hydrochloride, and the cortisol suppressant.

Bee pollen contains 22 amino acids, minerals, vitamins, and enzymes, and functions as an energy booster. Bee pollen also provides anti-aging and weight control benefits and may also serve as a natural antidepressant.

L-tyrosine is a nonessential amino acid that impacts the structure of almost all proteins in the body. As a precursor to the neurotransmitters of norepinephrine and epinephrine, which are stress-related hormones in the body, L-tyrosine may mitigate adverse effects of environmental, psychosocial, and physical stress.

Taurine is an amino acid that supports the healing of muscle tissue. Additionally, taurine functions in electrically active tissues such as the brain and heart to help stabilize cell membranes. Moreover, taurine plays a role in the gallbladder, eyes, and blood vessels (i.e., acts as a vaso-dilator) and promotes antioxidant and detoxifying activity. Taurine also facilitates migration of potassium, sodium, calcium, and magnesium in and out of cells so as to assist in the generation of nerve impulses.

D-ribose is a simple carbohydrate molecule that facilitates the continuous production of adenosine triphosphate (ATP), and ATP provides necessary energy to the muscles and the heart. Adding D-ribose to the nutritional supplement of the present invention increases energy levels within the muscle cell, enhances muscular performance, and promotes muscular recovery after high intensity exercise.

Colustrum builds and strengthens muscle mass, and increases both energy and stamina. In addition, colustrum is rich in broad-spectrum immune system boosters and natural growth factors.

Whey protein is a natural, high quality protein that includes all of the essential amino acids needed by the body on a daily basis and is very easy to digest (i.e., is assimilated rapidly into the bloodstream). Unlike other amino acids that are metabolized into the liver, whey protein is metabolized directly into muscle tissue. Thus, whey protein is ideal for helping the body to quickly repair and rebuild muscular tissue following muscular exercise. Useful forms of whey protein include whey protein concentrate, whey protein isolate, and hydrolyzed whey protein.

Lycopene is the major carotenoid in tomatoes and is responsible for the red color in fruits. Nutritionally, Lycopene is an antioxidant that may lower the risk of cancer and heart attack.

Glutamine stimulates the production of human growth hormone. Human growth hormone stimulates the growth of muscle mass and assists the body in burning fat, as explained supra. Glutamine also contributes directly to the building of muscle tissue by generating a hydrated state within the cell. The hydrated state of the cell facilitates an increase of nucleic acid and protein synthesis, and also facilitates the prevention of protein breakdown.

Macuna pruriens is a legume containing about 15% L-Dopa (l-dihydroxyphenylalanine). L-Dopa is converted to dopamine in the body and acts on the brain to increase the release of human growth hormone.

Alpha GPC is derived from soy lecithin and acts as a precursor to aceetylcholine, increasing human growth secretion by inhibiting somatostatin (a chemical in the brain that disables the release of human growth hormone).

Note that it is within the scope of the present invention for the nutritional supplement to not include a steroid and/or to not include a hormone.

In second embodiments, the nutritional supplement may consist essentially of: L-arginine-2-pyrrolidone-5-carboxylate in an amount of about 500 milligrams to about 10 grams; L-lysine hydrochloride in an amount of about 500 milligrams to about 10 grams; and a cortisol suppressant comprising at least one of acetyl-L-carnitine in an amount of about 1 gram to about 10 grams and maltodextrin in an amount of about 1 gram to about 10 grams. In accordance with the “consist essentially of” language, the nutritonal supplement of the second embodiments is essentially limited to the aforementioned ingredients and does not include any additional ingredients intended to add nutritional content (e.g., vitamins, minerals, etc.), but may include additional ingredients not intended to add nutritional content such as ingredients intended to fulfill a non-nutritional purpose (e.g., coloring, flavoring, an ingredient such as a filler for maintaining the structural form, etc.).

In third embodiments, the nutritional supplement may consist essentially of:

L-arginine-2-pyrrolidone-5-carboxylate in an amount of about 500 milligrams to about 10 grams;

L-lysine hydrochloride in an amount of about 500 milligrams to about 10 grams;

a cortisol suppressant including at least one of acetyl-L-carnitine in an amount of about 1 gram to about 10 grams and maltodextrin in an amount of about 1 gram to about 10 grams; and

a substance consisting essentially of at least one of:
L-leucine in an amount of about 500 milligrams to about 10 grams,
bee pollen in an amount of about 50 milligrams to about 10 grams,
L-tyrosine in an amount of about 500 milligrams to about 10 grams,
taurine in an amount of about 500 milligrams to about 10 grams,
D-ribose in an amount of about 50 milligrams to about 10 grams,
colustrum in an amount of about 50 milligrams to about 10 grams,
whey protein in an amount of about 1 gram to about 20 grams,
lycopene in an amount of about 100 milligrams to about 10 grams,
glutamine in an amount of about 500 milligrams to about 10 grams, and
both macuna pruriens and alpha glycerylphosphorylcholine (GPC), said macuna pruriens in an amount of about 50 milligrams to about 5 grams, said alpha GPC in an amount of about 50 milligrams to about 5 grams.

In accordance with the “consist essentially of” and “consisting essentially of” language, the nutritonal supplement of the third embodiments is essentially limited to the aforementioned ingredients and does not include any additional ingredients intended to add nutritional content (e.g., vitamins, minerals, etc.), but may include additional ingredients not intended to add nutritional content such as ingredients intended to fulfill a non-nutritional purpose (e.g., coloring, fillers, flavoring, an ingredient for maintaining the structural form, etc.).

Each ingredient of the nutritional supplement of the present invention may be prepared in accordance with any method known to one of ordinary skill in the art. Alternatively, each ingredient may be obtained in a fully prepared from a commercially available source.

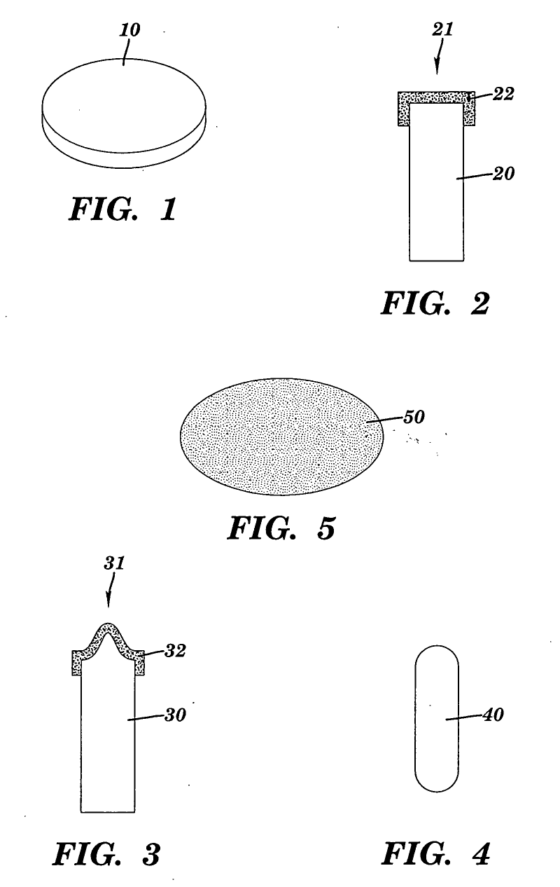

The nutritional supplement of the present invention may be in any of the following structural forms: a chewable form, a liquid form, a spray form, a capsule form, a suppository form, and a powder form, as described next in conjunction with FIGS. 1-5.

FIG. 1 depicts chewable form 10, such as a wafer or a chewable pill. Although FIG. 1 shows the chewable form 10 as being round, the chewable form may have any geometric shape, (e.g., circular, elliptical, rectangular square, triangular, etc.). The size of the chewable form 10 is of a size sufficient to contain all ingredients of the nutritional supplement in the amounts desired. For example, the chewable form 10 may be, inter alia, circular with a diameter of 1½ inches and a thickness of ½ inch. The chewable form 10 is adapted to be chewed by a person for easy and quick ingestion. The chewable form may be prepared by any method known to one of ordinary skill in the art for preparing such chewable forms.

The structural form of the present invention may comprise a dissolvable form such as a dissolvable wafer or dissolvable pill, which may be constituted structurally similar to the chewable form 10 shown in FIG. 1. The dissolvable form may dissolve in the mouth of the person when held under the person's tongue, for subsequent absorption into the bloodstream of the person. This dissolvable form may be prepared by any method known to one of ordinary skill in the art for preparing such dissolvable forms.

FIG. 2 depicts the liquid form 20 contained in a vial or equivalent container 21 having a top 22. The top 22 may be, inter alia, a screwable top, a puncturable top, etc. The liquid form 20 is adapted to be swallowed by the person for subsequent ingestion. The liquid form 20 may alternatively be adapted to be introduced into the body of the person by injection through use of a needle or syringe (at any suitable point of injection on the person's body). The liquid form may be prepared by any method known to one of ordinary skill in the art for preparing such liquid forms.

The structural form of the present invention may comprise an alternative dissolvable form such as a liquid, which may be constituted structurally similar to the liquid form 20 form shown in FIG. 1. The dissolvable form may dissolve in the mouth of the person when held under the person's tongue, for quick absorption into the bloodstream of the person. This dissolvable form may be prepared by any method known to one of ordinary skill in the art for preparing such dissolvable forms.

FIG. 3 depicts the spray form 30 contained in a spray bottle or equivalent container 31 having a cover 32. The cover 32 may be, inter alia, a screwable cover. The spray form 30 may be any type of spray (e.g., aerosol spray in a pressurized container 31) as is known in the art. The spray form 30 is adapted to be sprayed into the nasal cavity of the person for subsequent absorption into the bloodstream of the person. The spray form may be prepared by any method known to one of ordinary skill in the art for preparing such spray forms.

FIG. 4 depicts a capsule form 40, which is intended to be swallowed for subsequent ingestion and absorption into the bloodstream of the person. The capsule form may be prepared by any method known to one of ordinary skill in the art for preparing such capsule forms.

The structural form of the present invention may comprise a suppository form, which may be constituted structurally similar to the capsule form 40 shown in FIG. 4. The suppository form is intended for insertion into the anal cavity of the person for subsequent absorption into the bloodstream of the person. The suppository form may be prepared by any method known to one of ordinary skill in the art for preparing such suppository forms.

FIG. 5 depicts a powder form 50 which may be mixed with, or dissolved in, a liquid such as water or fruit juice. The liquid is to be subsequently swallowed by the person, so that the powder 50 therein will be absorbed into the bloodstream of the person. The powder 50 may alternatively be introduced into the body of the person by any other means such as by being mixed with applesauce, such that the applesauce with the powder 50 mixed therein is subsequently ingested by the person. The powder form may be prepared by any method known to one of ordinary skill in the art for preparing such powder forms.

Irrespective of the structural form of the nutritional supplement, the ingredients of the nutritional supplement may be distributed homogeneously or inhomogeneously within the nutritional supplement.

The nutritional supplement of the present invention may be ingested on a regular basis, such as a daily intake at a dosage tailored to an individual's needs; i.e., the nutritional supplement is to be taken regularly as multiples (1×, 2×, etc.) of the structural units (pills, tablets, capsules, etc.) in accordance with the needs of the individual. For example, a young person engaged in regular strenuous exercise (e.g., a weight lifter) is likely to need higher daily doses than does a senior citizen leading a sedentary life. Alternatively, the nutritional supplement of the present invention may be ingested on an as-needed basis at a dosage tailored to the individual's needs. Medical or nutritional counseling may be beneficial for arriving at a desirable or optimal dosage tailored to the individual's needs.

While embodiments of the present invention have been described herein for purposes of illustration, many modifications and changes will become apparent to those skilled in the art. Accordingly, the appended claims are intended to encompass all such modifications and changes as fall within the true spirit and scope of this invention.

1. A nutritional supplement, comprising:
L-arginine-2-pyrrolidone-5-carboxylate in an amount of about 500 milligrams to about 10 grams;
L-lysine hydrochloride in an amount of about 500 milligrams to about 10 grams; and
a cortisol suppressant including at least one of acetyl-L-carnitine in an amount of about 1 gram to about 10 grams and maltodextrin in an amount of about 1 gram to about 10 grams.

2. The nutritional supplement of claim 1, wherein the cortisol suppressant includes the acetyl-L-carnitine.

3. The nutritional supplement of claim 1, wherein the cortisol suppressant includes the maltodextrin.

4. The nutritional supplement of claim 1, wherein the cortisol suppressant includes the acetyl-L-carnitine and the maltodextrin.

5. The nutritional supplement of claim 1, further comprising L-leucine in an amount of about 500 milligrams to about 10 grams.

6. The nutritional supplement of claim 1, further comprising bee pollen in an amount of about 50 milligrams to about 10 grams.

7. The nutritional supplement of claim 1, further comprising L-tyrosine in an amount of about 500 milligrams to about 10 grams.

8. The nutritional supplement of claim 1, further comprising taurine in an amount of about 500 milligrams to about 10 grams.

9. The nutritional supplement of claim 1, further comprising D-ribose in an amount of about 50 milligrams to about 10 grams.

10. The nutritional supplement of claim 1, further comprising colustrum in an amount of about 500 milligrams to about 10 grams.

11. The nutritional supplement of claim 1, further comprising whey protein in an amount of about 1 gram to about 20 grams.

12. The nutritional supplement of claim 1, further comprising lycopene in an amount of about 100 milligrams to about 10 grams.

13. The nutritional supplement of claim 1, further comprising glutamine in an amount of about 500 milligrams to about 10 grams.

14. The nutritional supplement of claim 1, further comprising macuna pruriens in an amount of about 50 milligrams to about 5 grams and alpha glycerylphosphorylcholine in an amount of about 50 milligrams to about 5 grams.

15. The nutritional supplement of claim 1, wherein the nutritional supplement does not include a steroid, and wherein the nutritional supplement does not include a hormone.

16. The nutritional supplement of claim 1, wherein the nutritional supplement is in a powder form.

17. The nutritional supplement of claim 1, wherein the nutritional supplement is in a tablet form.

18. The nutritional supplement of claim 1, wherein the nutritional supplement is in a wafer form.

19. The nutritional supplement of claim 1, wherein the nutritional supplement is in a liquid form.

20. The nutritional supplement of claim 1, wherein the nutritional supplement is in a capsule form.

21. A nutritional supplement consisting essentially of:
L-arginine-2-pyrrolidone-5-carboxylate in an amount of about 500 milligrams to about 10 grams;
L-lysine hydrochloride in an amount of about 500 milligrams to about 10 grams; and
a cortisol suppressant including at least one of acetyl-L-carnitine in an amount of about 1 gram to about 10 grams and maltodextrin in an amount of about 1 gram to about 10 grams.

22. The nutritional supplement of claim 21, wherein the nutritional supplement consists essentially of the L-arginine-2-pyrrolidone-5-carboxylate, the L-lysine hydrochloride, and the maltodextrin.

23. The nutritional supplement of claim 21, wherein the nutritional supplement is in a powder form, and wherein the nutritional supplement consists essentially of the L-arginine-2-pyrrolidone-5-carboxylate in an amount of about 1.5 grams, the L-lysine hydrochloride in an amount of about 1.5 grams, and the maltodextrin in an amount of about 3 grams.

24. The nutritional supplement of claim 21, wherein the cortisol suppressant includes the acetyl-L-carnitine.

25. The nutritional supplement of claim 21, wherein the cortisol suppressant includes the maltodextrin.

26. The nutritional supplement of claim 21, wherein the cortisol suppressant includes the acetyl-L-carnitine and the maltodextrin.

27. The nutritional supplement of claim 21, wherein the nutritional supplement is in a powder form.

28. The nutritional supplement of claim 21, wherein the nutritional supplement is in a tablet form.

29. The nutritional supplement of claim 21, wherein the nutritional supplement is in a wafer form.

30. The nutritional supplement of claim 21, wherein the nutritional supplement is in a liquid form.

31. The nutritional supplement of claim 21, wherein the nutritional supplement is in a capsule form.

32. A nutritional supplement consisting essentially of:
L-arginine-2-pyrrolidone-5-carboxylate in an amount of about 500 milligrams to about 10 grams;
L-lysine hydrochloride in an amount of about 500 milligrams to about 10 grams;
a cortisol suppressant including at least one of acetyl-L-carnitine in an amount of about 1 gram to about 10 grams and maltodextrin in an amount of about 1 gram to about 10 grams; and
a substance consisting essentially of at least one of:
L-leucine in an amount of about 500 milligrams to about 10 grams,
bee pollen in an amount of about 50 milligrams to about 10 grams,
L-tyrosine in an amount of about 500 milligrams to about 10 grams,
taurine in an amount of about 500 milligrams to about 10 grams,
D-ribose in an amount of about 50 milligrams to about 10 grams,
colustrum in an amount of about 50 milligrams to about 10 grams,
whey protein in an amount of about 1 gram to about 20 grams,
lycopene in an amount of about 100 milligrams to about 10 grams,
glutamine in an amount of about 500 milligrams to about 10 grams, and
both macuna pruriens and alpha glycerylphosphorylcholine (GPC), said macuna pruriens in an amount of about 50 milligrams to about 5 grams, said alpha GPC in an amount of about 50 milligrams to about 5 grams.

33. The nutritional supplement of claim 32, wherein the substance includes the L-leucine.

34. The nutritional supplement of claim 32, wherein the substance includes the bee pollen.

35. The nutritional supplement of claim 32, wherein the substance includes the L-tyrosine.

36. The nutritional supplement of claim 32, wherein the substance includes the taurine.

37. The nutritional supplement of claim 32, wherein the substance includes the D-ribose.

38. The nutritional supplement of claim 32, wherein the substance includes the colustrum.

39. The nutritional supplement of claim 32, wherein the substance includes the whey.

40. The nutritional supplement of claim 32, wherein the substance includes the lycopene.

41. The nutritional supplement of claim 32, wherein the substance includes the glutamine.

42. The nutritional supplement of claim 32, wherein the substance includes the macuna pruriens and alpha GPC.

43. The nutritional supplement of claim 32, wherein the substance consists essentially of the L-leucine.

44. The nutritional supplement of claim 32, wherein the substance consists essentially of the bee pollen.

45. The nutritional supplement of claim 32, wherein the substance consists essentially of the L-tyrosine.

46. The nutritional supplement of claim 32, wherein the substance consists essentially of the taurine.

47. The nutritional supplement of claim 32, wherein the substance consists essentially of the D-ribose.

48. The nutritional supplement of claim 32, wherein the substance consists essentially of the colustrum.

49. The nutritional supplement of claim 32, wherein the substance consists essentially of the whey.

50. The nutritional supplement of claim 32, wherein the substance consists essentially of the lycopene.

51. The nutritional supplement of claim 32, wherein the substance consists essentially of the glutamine.

52. The nutritional supplement of claim 32, wherein the substance consists essentially of the macuna pruriens and alpha GPC.

53. The nutritional supplement of claim 32, wherein the nutritional supplement is in a powder form.

54. The nutritional supplement of claim 32, wherein the nutritional supplement is in a tablet form.

55. The nutritional supplement of claim 32, wherein the nutritional supplement is in a wafer form.

56. The nutritional supplement of claim 32, wherein the nutritional supplement is in a liquid form.

57. The nutritional supplement of claim 32, wherein the nutritional supplement is in a capsule form.
